(19) United States
(12) Patent Application Publication
(10) Pub. No.: US 2019/0076828 A1
(43) Pub. Date: Mar. 14, 2019

(54) CATALYSTS AND METHODS FOR METHANOL SYNTHESIS FROM DIRECT HYDROGENATION OF SYNGAS AND/OR CARBON DIOXIDE

(71) Applicant: SABIC Global Technologies B.V., Bergen op Zoom (NL)

(72) Inventors: Khalid A. ALMUSAITEER, Thuwal (SA); Ahmed AL-HADHRAMI, Thuwal (SA); Omar ABED, Thuwal (SA); Gregory BIAUSQUE, Thuwal (SA); Abdulaziz AL-AMER, Thuwal (SA)

(21) Appl. No.: 16/093,790

(22) PCT Filed: Mar. 13, 2017
(86) PCT No.: PCT/IB2017/051450

§ 371 Date: Oct. 15, 2018

Related U.S. Application Data
(60) Provisional application No. 62/325,718, filed on Apr. 21, 2016.

(51) Int. Cl. B01J 23/843; B01J 23/83; B01J 23/835; B01J 23/80; B01J 35/00; B01J 35/02; B01J 37/03; C07C 29/156

(52) U.S. Cl. CPC B01J 23/8437; B01J 23/83; B01J 23/835; B01J 23/80; C07C 31/04; B01J 35/023; B01J 37/036; C07C 29/156; B01J 35/0013

(57) ABSTRACT

Nano-sized mixed metal oxide catalysts capable of producing methanol (CH₃OH) from carbon dioxide (CO₂) and hydrogen (H₂) or from carbon dioxide (CO₂), carbon monoxide (CO), and hydrogen (H₂), methods of making the catalyst, and uses thereof are described herein. The nano-sized mixed metal oxide catalysts can have a formula of: [CuₐZn_{b}Al_{c}M_{d}¹]Oₙ where a is 20 to 80, b is 15 to 60, c is 1 to 25, d is 0 to 15 and n is determined by the oxidation states of the other elements is determined by the oxidation states, and M¹ can be yttrium (Y), cerium (Ce), tin (Sn), sodium (Na), bismuth (Bi), magnesium (Mg), or gadolinium (Gd).

### CROSS REFERENCE TO RELATED APPLICATIONS

This application claims the benefit of priority of U.S. Provisional Patent Application No. 62/325,718 filed Apr. 21, 2016, which is hereby incorporated by reference in its entirety.

### BACKGROUND OF THE INVENTION

### A. Field of the Invention

The invention generally concerns catalysts capable of synthesizing alcohols from carbon dioxide and/or synthesis gas (syngas). In particular, a multicomponent heterogeneous catalyst composition containing mixed metal oxides is used to catalyze the direct hydrogenation of carbon dioxide and/or carbon monoxide to methanol. The catalysts show increased activity and selectivity compared to conventional catalysts under identical conditions.

### B. Description of Related Art

Carbon dioxide (CO₂) is mostly produced as a waste by-product in oil refinery, fossil fuels combustion, and chemicals production. Many natural gas sources contain sizeable concentrations (as much as 50%) of CO₂. Most of the CO₂ produced in above processes is released into the atmosphere. However, to mitigate CO₂ emissions and their adverse effects on the global climate, many efforts have been undertaken to develop new technologies and upgrade the current ones that would prevent or reduce CO₂ generation. In addition, capturing the generated CO₂ and using it for various applications such as in an enhanced oil recovery process or as an alternative feedstock and building block for several industrial chemicals has been investigated as an outlet for waste CO₂.

One method to use carbon dioxide is to produce methanol. As shown in reaction scheme (1), carbon dioxide can be hydrogenated in the presence of a copper catalyst to produce methanol.

CO₂+3H₂⇄CH₃OH+H₂O ΔH=−49.43 kJ/mol (1)

In this process, methanol formation is favored by lower temperature (less than 250° C.) and higher pressure using copper-based catalysts. At higher temperatures formation of other by-products besides methanol occurs, thus decreasing the amount of methanol formed. Further, deactivation of the copper catalysts can occur through formation of water on the active copper sites. Commercially, this problem has been addressed through the use of a two-step process referred to as the CAMERE process (carbon dioxide hydrogenation to form methanol via a reverse-water gas shift reaction or RWGSR). In the CAMERE process, two reactors are consecutively arranged to convert carbon dioxide to CO and H₂O in the first reactor by RWGSR (See, reaction scheme (2)). Water and, optionally, carbon dioxide can then be removed to form a stream rich in carbon monoxide. The enriched carbon monoxide stream can then be fed into the second reactor to produce methanol under catalytic conditions (See, reaction scheme (3)).

CO₂+2H₂→CO+2H₂O (2)

CO+2H₂→CH₃OH (3)

In this approach, RWGSR can be carried out at high temperature (>600° C.) under catalytic conditions to obtain high CO₂ conversion to CO. Conversion of CO to methanol in a second reactor can lead to high methanol productivity due to the removal of water. Other approaches to hydrogenate CO₂ include varying catalyst compositions, methods of preparation, and reaction conditions. By way of example, Chinese Patent Publication CN103721719 by Ning et al. describes a halogenated copper and mixed metal catalyst for the hydrogenation of carbon dioxide to methanol reaction. Chinese Patent Publication CN104549299 by Yang et al. describes a copper based catalyst with a surface metal promoter for use in the hydrogenation of carbon dioxide to methanol reactions. Korean Patent No. 1014476820000 describes a Cu/Zn/Mg/Al catalyst made under basic co-precipitation conditions for use in the hydrogenation of carbon dioxide to methanol reaction. U.S. Patent Application Publication No. 20110105306 describes 20110105306 to Chien et al. describes a Cu/Zn/Al catalyst on a support for the conversion of hydrogen and carbon dioxide to methanol followed by dehydration to produce dimethylether (DME). U.S. Pat. No. 8,999,881 to Budiman et al. describes a Cu/Zn/Al catalyst made under basic co-precipitation for the conversion of butyl butyrate to n-butanol. Still other approaches to increase methanol production include changing the type or combination of active components, supports, promoters, preparation methods, and surface morphology (See, for example, Gao et al. in American Chemical Society, Division of Fuel Chemistry (2012), 57(1), pp. 280-281 and Toyir et al. in Applied Catalysis B (2001), 29, pp. 207-215 and Applied Catalysis B (2001), 34, pp. 255-266).

Most of the above mentioned processes suffer from poor selectivity, increased formation of by-products, and decreased methanol yields or combinations thereof.

### SUMMARY OF THE INVENTION

A discovery has been made that solves the problems associated with the production of methanol from carbon dioxide. In particular, the discovery is premised on a mixed metal catalyst that includes various loadings of copper (Cu), zinc (Zn), aluminum (Al), and M¹ oxides. The M¹ oxide can include yttrium (Y), cerium (Ce), tin (Sn), sodium (Na), bismuth (Bi), magnesium (Mg), or gadolinium (Gd). These catalysts can be used to catalyze the direct hydrogenation of carbon dioxide (CO₂) and/or carbon monoxide (CO) to methanol (MeOH) in one pass. This eliminates the need for a multi-step reaction process such as those described above. The mixed metal catalysts of the present invention have shown increased methanol conversions with high single pass methanol molar flow in comparison to conventional catalysts in reactions carried out in fixed bed tubular reactors under H₂/CO/CO₂ (synthesis gas or syngas) or H₂/CO₂ flow at temperatures from 180 to 290° C. and pressures from 1.0 MPa to 10 MPa (10 to 100 bar). Notably, the catalysts can be used in the presence of excess amounts carbon dioxide (e.g., greater than 10 vol. % carbon dioxide added to, or present in, syngas). Without wishing to be bound by theory, it is believed that the combination of the metals in the catalysts of the present invention can promote oxygen storage and release, and also reduce or inhibit water from depositing on the active catalytic sites. Still further, it is believed that the presence of Y, Ce, Sn, Na, Bi, Mg, or Gd oxides increase the methanol yield due to a strong interaction with the CO and O atoms of CO₂ (due to large surface reconstruction). It was also discovered that preparation of the catalysts using a gel oxalate co-precipitation method provided nanoparticles of a desired shape and size that allow high CO₂ concentrations of up to 18% with marginal catalyst deactivation rate. The ability to increase the concentration of CO₂ in the syngas feed lowers the overall reaction exothermicity, resulting in less requirements for heat management.

In one aspect of the present invention, there is disclosed a mixed metal catalyst capable of producing methanol (CH₃OH) from hydrogen (H₂) and carbon dioxide (CO₂) or from hydrogen (H₂), carbon dioxide (CO₂) and carbon monoxide (CO), the catalyst having a general formula of:

[CuₐZn_{b}Al_{c}M_{d}¹]Oₙ

where a is 20 to 80, b is 15 to 60, c is 1 to 25, d is 0 to 15 and n is determined by the oxidation states of the other elements, and where M¹ is yttrium (Y), cerium (Ce), tin (Sn), sodium (Na), bismuth (Bi), magnesium (Mg) or gadolinium (Gd). Throughout the specification the [CuₐZn_{b}Al_{c}M_{d}¹]Oₙ can be referred to as [CuZnAlM¹]Oₙ. In instance when d is zero, the mixed metal catalyst can have a formula of: [CuₐZn_{b}Al_{c}]Oₙ. In preferred aspects, the mixed metal catalyst can be an oxalic acid coprecipitated catalyst. The mixed metal catalyst can have a particle size of 2 nm to 12 nm, or 8 nm as determined by X-ray diffraction, a Brunauer-Emmett-Teller (BET) surface area of 21 m²/g to 120 m²/g, a copper surface area of 12 m²/g to 38 m²/g a pore volume of 0.15 cm³/g to 4 cm³/g, a pore diameter of 10 nm to 18 nm, or any combination thereof. In a particular embodiment, the catalyst has particle size of 8 nm, a BET surface area of 70 m²/g, a copper surface area of 19.9 m²/g, a pore volume of 0.26 cm³/g, and a pore diameter of 14 nm. In some instances, the catalyst has initial crystalline phases of CuO and ZnO.

Also disclosed are methods of producing methanol (CH₃OH) from hydrogen (H₂) and carbon dioxide (CO₂) and and/or carbon monoxide (CO), the method can include contacting a reactant gas stream that includes H₂ and CO₂ and and/or CO with any of the mixed metal catalyst of the current invention under conditions sufficient to produce a product gas stream that can include CH₃OH. In one aspect, the reactant gas stream can include H₂ and CO₂. In another aspect, the reactant gas stream can include H₂, CO₂, and CO. The ratio of H₂/(CO₂+CO) can be 1.5 to 3.5, preferably 1.9 to 2.9. In some aspects, the reactant gas stream can include 30 to 80 vol. % H₂, 1 to 30 vol. % CO₂, and 0 to 60 vol. % CO. In other aspects, the reactant gas stream can include 1 vol. % to 20 vol. % CO₂, preferably 5 vol. % to 15 vol. % CO₂, and more preferably 8 vol. % to 12 vol. % CO₂. In any of the disclosed methods, CH₃OH can be produced in a single pass and the reaction conditions can include a temperature of 200° C. to 300° C., preferably 220° C. to 260° C., a pressure of 1 bar to 100 bar, preferably 50 bar to 90 bar, and a gas hourly space velocity of 2,500 h⁻¹ to 20,000 h⁻¹, preferably of 4,000 h⁻¹ to 10,000 h⁻¹. The methanol single space-time yield (STY) can be 600 g/L.cat.h to 900 g/L.cat.h at 200° C. to 260° C. and 40 bar to 100 bar, and the single pass CH₃OH selectivity can be 40% to 100%, preferably, 50% to 90%, or more preferably from 60% to 80% after 300 hours TOS. In a particular aspect, the single pass CO₂ conversion is 20% to 35% at 2200° C. to 260° C. and 4 MPa to 10 MPa (40 bar to 100 bar).

Also disclosed are methods of making a mixed metal oxide catalyst of the current invention. A method can include (a) obtaining a first solution containing metal precursor materials that includes copper (Cu), zinc (Zn), aluminum (Al) and, optionally M¹, where M¹ is yttrium (Y), cerium (Ce), tin (Sn), magnesium (Mg), sodium (Na), bismuth (Bi), gadolinium (Gd), or any combination thereof dissolved in alcohol, (b) obtaining a second solution containing oxalic acid dissolved in an alcohol, (c) mixing the first and second solution together to form a precipitate from the metal precursor materials, and (d) calcining the precipitate to obtain the mixed metal oxide catalyst. In one aspect, the precipitate obtained in step (c) can be dried at 90° C. to 120° C.; and subsequently calcined in step (d) for 2 to 6 hours at a temperature of 250° C. to 450° C. In certain instances, the metal precursor material includes M¹.

In another instances, a method of making a mixed metal oxide catalyst of the present invention includes a glycolic acid co-precipitation method. The method can include (a) obtaining an aqueous or alcoholic solution that includes a metal precursor material that includes copper (Cu), zinc (Zn), aluminum (Al) and, optionally, M¹, where M¹ is yttrium (Y), cerium (Ce), tin (Sn), sodium (Na), bismuth (Bi), magnesium (Mg), gadolinium (Gd), or any combination thereof, wherein the metal precursors are dissolved in the aqueous solution; (b) adding a precipitating agent acid to the solution in step (a); (c) heating the solution to form a gel; and (d) drying and calcining the gel to obtain the mixed metal oxide catalyst of any one of claims 1 to 10. In some instances, the precipitating agent is glycolic acid or a diamine, for example, ethylene diamine. During, or after, the addition of the precipitating agent (e.g., glycolic acid) to the solution in step (a), the pH of the solution can be adjusted to 7.0 to 8.0, preferably 7.2 to 7.5. In certain instances, the metal precursor material includes M¹.

In certain instances, a method of making the mixed metal oxide catalyst can include making an alcoholic mixture of the metal precursor material and oxalic acid. Such a method can include (a) mixing oxalic acid, an alcohol, and metal precursor materials to form a precipitate, wherein the metal precursor materials include copper (Cu), zinc (Zn), aluminum (Al) and, optionally, M¹, where M¹ is yttrium (Y), cerium (Ce), tin (Sn), sodium (Na), bismuth (Bi), magnesium (Mg), gadolinium (Gd), or any combination thereof; and (b) drying and calcining the precipitate to obtain the mixed metal oxide catalyst of any one of claims 1 to 10. In some instances, the metal precursor materials include M¹.

The following includes definitions of various terms and phrases used throughout this specification.

The term “mixed metal oxide” catalyst refers to a catalyst that can include metals substantially as oxides or a mixture of metal oxides and metals in other forms (e.g., reduced metal form).

The term “bulk metal oxide catalyst” or “bulk mixed metal oxide catalyst” as that terms are used in the specification and/or claims, means that the catalyst includes metals, and does not require a carrier or a support.

The term “conversion” means the mole fraction (i.e., percent) of a reactant converted to a product or products.

The term “selectivity” refers to the percent of converted reactant that went to a specified product, for example methanol selectivity is the % of CO₂ that formed methanol.

The term “about” or “approximately” are defined as being close to as understood by one of ordinary skill in the art. In one non-limiting embodiment the terms are defined to be within 10%, preferably within 5%, more preferably within 1%, and most preferably within 0.5%.

The terms “wt. %”, “vol. %”, or “mol. %” refers to a weight, volume, or molar percentage of a component, respectively, based on the total weight, the total volume of material, or total moles, that includes the component. In a non-limiting example, 10 grams of component in 100 grams of the material is 10 wt. % of component.

The term “substantially” and its variations are defined to include ranges within 10%, within 5%, within 1%, or within 0.5%.

The terms “inhibiting” or “reducing” or “preventing” or “avoiding” or any variation of these terms, when used in the claims and/or the specification includes any measurable decrease or complete inhibition to achieve a desired result.

The term “effective,” as that term is used in the specification and/or claims, means adequate to accomplish a desired, expected, or intended result.

The use of the words “a” or “an” when used in conjunction with any of the terms “comprising,” “including,” “containing,” or “having” in the claims, or the specification, may mean “one,” but it is also consistent with the meaning of “one or more,” “at least one,” and “one or more than one.”

The words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps.

The catalysts and methods of the present invention can “comprise,” “consist essentially of,” or “consist of” particular ingredients, components, compositions, etc. disclosed throughout the specification. With respect to the transitional phase “consisting essentially of,” in one non-limiting aspect, a basic and novel characteristic of the catalysts of the present invention are their ability to catalyze the direct hydrogenation of carbon dioxide and carbon dioxide/carbon monoxide mixtures to produce methanol.

Other objects, features and advantages of the present invention will become apparent from the following figures, detailed description, and examples. It should be understood, however, that the figures, detailed description, and examples, while indicating specific embodiments of the invention, are given by way of illustration only and are not meant to be limiting. Additionally, it is contemplated that changes and modifications within the spirit and scope of the invention will become apparent to those skilled in the art from this detailed description.

### BRIEF DESCRIPTION OF THE DRAWINGS

FIG. 1 is a schematic of an embodiment of a system for producing methanol from synthesis gas.

FIG. 2 shows a graphical representation of a methanol space-time yield (STY) as a function of time on stream (TOS) during the CO₂ addition into H₂/CO mixture at 240° C., 40 bar and 5000 h⁻¹ of Cu/Zn/Al, Cu/Zn/Al/Y and Cu/Zn/Al/Ce metal oxide catalysts of the present invention and a comparative catalyst.

FIG. 3 shows a graphical representation of CO molar flow rate as a function of time on stream (TOS) during the CO₂ addition into H₂/CO mixture at 240° C., 40 bar and 5000 h⁻¹ with Cu/Zn/Al, Cu/Zn/Al/Y and Cu/Zn/Al/Ce metal oxide catalysts and a comparative catalyst.

FIG. 4 shows a graphical representation of H₂/(CO₂+CO) molar ratio as a function of time on stream (TOS) during the CO₂ addition into H₂/CO mixture at 240° C., 40 bar and 5000 h⁻¹ from FIG. 3.

FIG. 5 shows a graphical representation of a methanol space-time yield (STY) as a function of time on stream (TOS) during the CO₂ addition into H₂/CO mixture at 240° C., 40 bar and 5000 h⁻¹ of Cu/Zn/Al/Sn and Cu/Zn/Al/Mg metal oxide catalysts of the present invention.

FIG. 6 shows the molar flow rate of methanol from hydrogenation of CO₂ and after the addition of 14 vol. % CO₂ into a H₂/CO mixture at 240° C., 4.0 MPa (40 bar) and 5000 h⁻¹ over various Cu/Zn/Al nano-sized catalysts prepared using different alcohols and the commercial catalyst.

FIG. 7 shows the molar flow rate of carbon monoxide from hydrogenation of CO₂ and after the addition of 14 vol. % CO₂ into a H₂/CO mixture at 240° C., 40 bar and 5000 h⁻¹ over various Cu/Zn/Al nano-sized catalysts prepared using different alcohols and the commercial catalyst.

FIG. 8 shows the molar flow rate of carbon monoxide from hydrogenation of CO₂ and after the addition of 14 vol. % CO₂ into a 55 Vol. % H₂/11 vol. % CO mixture at 220° C. to 260° C., 4.0 MPa (40 bar) and 5000 h⁻¹ over Cu/Zn/Al nano-sized catalysts prepared at different calcination temperatures and two commercial catalysts.

FIG. 9 is an X-Ray Diffraction pattern of a Cu/Zn/Al catalyst of the present invention.

FIG. 10 shown X-Ray Diffraction patterns of various catalysts of the present invention.

### DETAILED DESCRIPTION OF THE INVENTION

A discovery has been made, which provides stable, highly active nano-sized catalysts for the direct hydrogenation of synthesis gas (syngas) and CO/CO₂ mixtures to methanol in one pass. The discovery is premised on the use of nano-sized mixed metal catalysts prepared by gel oxalate co-precipitation that show high resistance to water. In particular, the discovery is premised on the use of various loadings of nano-particles of copper (Cu), zinc (Zn), aluminum (Al), and M¹ (e.g., Y, Ce, Sn, Na, Mg, Bi, or Gd). Without wishing to be bound by theory, it is believed that the selected combination of metals can promote oxygen storage and release, and reduce or inhibit water from depositing on the active catalytic sites. The invention provides an elegant way to provide a cost-effective methods to directly hydrogenate CO₂ and/or CO₂/CO mixtures having more than 10 wt. % CO₂, thereby providing a solution to environmental issues concerning the use or disposal of CO₂. The ability to increase the concentration of CO₂ in the syngas feed lowers the overall reaction exothermicity, resulting in less requirements for heat management.

These and other non-limiting aspects of the present invention are discussed in further detail in the following sections.

### A. Mixed Metal Oxide Catalyst

The nano-sized mixed metal catalysts of the present invention are capable of direct hydrogenation of carbon dioxide and hydrogen or mixtures of carbon dioxide and carbon monoxide to produce methanol in a single pass. One or more of these nano-sized mixed metal catalysts can include a heterogeneous mixture of metals (e.g., metals in reduced form), metal compounds (e.g., metal oxides) or mixtures thereof (“collectively metals”) of alkali metals, alkali earth metals, transition metals, post-transition metals, and lanthanides of the Periodic Table. The metals in the nano-sized mixed metal catalyst can exist in one or more oxidation states. Non-limiting examples of alkali and alkali earth metals includes lithium (Li), sodium (Na), rubidium (Rb), magnesium (Mg), barium (Ba) and strontium (Sr). Non-limiting examples of transition metals include yttrium (Y), titanium (Ti), zirconium (Zr), molybdenum (Mo), tungsten (W), copper (Cu), silver (Ag), and zinc (Zn). Non-limiting examples of post-transition metals include aluminum (Al), gallium (Ga), tin (Sn), and bismuth (Bi). Non-limiting examples of the lower lanthanides include lanthanum (La), cerium (Ce), gadolinium (Gd), and terbium (Tb). Preferably the mixed metal oxide nano-sized catalyst contains copper, zinc, aluminium, and M¹, where M¹ is yttrium, cerium, tin, magnesium, sodium, bismuth, gadolinium, or any combination thereof. The catalysts of the present invention do not include a support (e.g., gamma alumina, silicon dioxide, titanium dioxide or the like). The catalyst can have an atomic ratio of metals ranging from about 1 to about 99. For example, in one aspect the atomic ratio of a Cu/Zn/Al/Y catalyst can range from about 20-80:15-60:1-25:0-15, or 40-75:20-50:1-10:0-10, preferably about 60:30:10:0, about 60:30:9:1, about 60:30:8:2, about 60:30:7:3, about 60:30:6:4, and about 60:30:5:5. In another aspect the atomic ratio of a Cu/Zn/Al/Ce catalyst can range from about 20-80:15-60:1-25:0-15, or 40-75:20-50:1-10:0-10, preferably about 60:30:10:0, about 60:30:9:1, about 60:30:8:2, about 60:30:7:3, about 60:30:6:4, and about 60:30:5:5. In another aspect the atomic ratio of a Cu/Zn/Al/Sn catalyst can range from about 20-80:15-60:1-25:0-15, or 40-75:20-50:1-10:0-10, preferably about 60:30:10:0, about 60:30:5:1, about 60:30:5:2, about 60:30:5:3, about 60:30:5:4, and about 60:30:5:5. In another aspect the atomic ratio of a Cu/Zn/Al/Mg catalyst can range from about 20-80:15-60:1-25:0-15, or 40-75:20-50:1-10:0-10, preferably about 60:30:10:0, about 60:30:9:1, about 60:30:8:2, about 60:30:7:3, about 60:30:6:4, and about 60:30:5:5. In another aspect the atomic ratio of a Cu/Zn/Al/Na catalyst can range from about 20-80:15-60:1-25:0-15, or 40-75:20-50:1-10:0-10, preferably about 60:30:10:0, about 60:30:5:1, about 60:30:5:2, about 60:30:5:3, about 60:30:5:4, and about 60:30:5:5. In still another aspect the atomic ratio of a Cu/Zn/Al/Bi catalyst can range from about 20-80:15-60:1-25:0-15, or 40-75:20-50:1-10:0-10, preferably about 60:30:10:0, about 60:30:9:1, about 60:30:8:2, about 60:30:7:3, about 60:30:6:4, and about 60:30:5:5. In yet another aspect the atomic ratio of a Cu/Zn/Al/Gd catalyst can range from about 20-80:15-60:1-25:0-15, or 40-75:20-50:1-10:0-10, preferably about 60:30:10:0, about 60:30:9:1, about 60:30:8:2, about 60:30:7:3, about 60:30:6:4, and about 60:30:5:5. In embodiments when the Al or Y value is greater than 1, the above values have not been normalized. It should be understood that a, for example, 20:15:5 ratio is the same as 4:3:1. Copper loading in the catalyst can be from 20 mole % to about 80 mole %, from about 30 mole % to about 70 mole %, and preferably from about 40 mole % to about 60 mole %. Zinc loading in the catalyst can be from 15 mole % to about 60 mole %, from about 20 mole % to about 40 mole %, and preferably from about 25 mole % to about 35 mole %. Aluminum loading in the catalyst can be from 1 mole % to about 25 mole %, from about 5 mole % to about 15 mole %, and preferably from about 5 mole % to about 10 mole %. In some instances, the mixed metal oxide catalysts of the current invention do not include boron (B), silicon (Si), or a halogen (F, Cl, Br, or I). The metals used to prepare the catalyst of the present invention can be provided in varying oxidation states as metallic, oxide, hydrate, or salt forms typically depending on the propensity of each metals stability, reactivity, and/or physical/chemical properties. The metals or metal oxides used in the preparation of the mixed metal oxide catalyst can be provided in stable oxidation states as complexes with monodentate, bidentate, tridentate, or tetradendrate coordinating ligands such as for example iodide, bromide, sulfide, thiocyanate, chloride, nitrate, azide, acetate, fluoride, hydroxide, oxalate, water, isothiocyanate, acetonitrile, pyridine, ammonia, ethylenediamine, 2,2′-bipyridine, 1,10-phenanthroline, nitrite, triphenylphosphine, cyanide, or carbon monoxide. In some embodiments, the mixed metal oxides used to prepare the catalysts of the current invention can be provided as acetate, nitrate, nitrate hydrates, nitrate trihydrates, nitrate pentahydrate, nitrate hexahydrates, and nitrate nonahydrate, for example, copper (II) nitrate trihydrate, zinc nitrate hexahydrate, aluminum nitrate nonahydrate, yttrium (III) nitrate hexahydrate, cerium (III) nitrate hexahydrate, tin (II) acetate, magnesium nitrate hexahydrate, sodium nitrate, bismuth (III) nitrate pentahydrate, and gadolinium (III) nitrate hexahydrate. Various commercial sources can be used to obtain the metals and metal oxides. A non-limiting example of a commercial source of the above mentioned metals and metal oxides is Sigma Aldrich® (U.S.A.).

The nano-sized catalytic materials of the present invention have physical properties that can contribute to the catalytic properties and stability of the catalyst in the direct hydrogenation of carbon dioxide and/or mixtures of carbon dioxide and carbon monoxide to methanol. The catalyst can have a particle size of 2 to 12 nm or 5 nm to 10 nm, or 2 nm, 3 nm, 4 nm, 5 nm, 6 nm, 7 nm, 8 nm, 9 nm, 10 nm, 11 nm, 12 nm or any range or value there between. The BET surface area of the catalyst can range from 21 m²/g to 120 m²/g, 30 m²/g to 80 m²/g, 50 m²/g to 70 m²/g, or 21 m²/g, 25 m²/g, 30 m²/g, 35 m²/g, 40 m²/g, 45 m²/g, 50 m²/g, 55 m²/g, 60 m²/g, 65 m²/g, 70 m²/g, 75 m²/g, 80 m²/g, 85 m²/g, 90 m²/g, 95 m²/g, 100 m²/g, 110 m²/g, 115 m²/g, 120 m²/g, or any value or range there between. The pore volume of the catalyst can range from 0.15 cm³/g to 4 cm³/g, 0.2 cm³/g to 3 cm³/g, 0.5 to 1 cm³/g, or 0.15 cm³/g, 0.2 cm³/g, 0.25 cm³/g, 0.3 cm³/g, 0.35 cm³/g, 0.4 cm³/g, 0.45 cm³/g, 0.50 cm³/g, 0.55 cm³/g, 0.60 cm³/g, 0.65 cm³/g, 0.70 cm³/g, 0.75 cm³/g, 0.80 cm³/g, 0.85 cm³/g, 0.90 cm³/g, 0.95 cm³/g, 1.0 cm³/g, 1.5 cm³/g, 2.0 cm³/g, 2.5 cm³/g, 3.0 cm³/g, 3.5 cm³/g, 4.0 cm³/g or any value or range there between. The pore diameter of the catalyst particle can range from 10 nm to 18 nm, 12 nm to 15 nm, or 10 nm, 11 nm, 12 nm, 13 nm, 14 nm, 15 nm, 16 nm, 17 nm, 18 nm, or any value or range there between. The average CuO particle size in the catalyst can range from 1 to 12 nm. The mixed metal oxide catalyst can include copper in the Cu⁰ and Cu⁺¹ oxidation states. The BET surface area of the copper species can range from 12 m²/g to 38 m²/g, or 15 m²/g to 25 m²/g, or 12 m²/g, 13 m²/g, 14 m²/g, 15 m²/g, 16 m²/g, 17 m²/g, 18 m²/g, 19 m²/g, 20 m²/g, 21 m²/g, 22 m²/g, 23 m²/g, 24 m²/g, 25 m²/g, 26 m²/g, 27 m²/g, 28 m²/g, 29 m²/g, 30 m²/g, 31 m²/g, 32 m²/g, 33 m²/g, 34 m²/g, 35 m²/g, 36 m²/g, 37 m²/g, 38 m²/g or any range or value there between. Without wishing to be bound by theory, it is believed that a the properties of the catalysts allows absorption of the carbon dioxide, carbon monoxide, and hydrogen on the catalytic surface, thereby improving the proximity of hydrogen to carbon dioxide/carbon monoxide and reducing the production of by-products during direct hydrogenation of carbon dioxide to methanol reaction.

### B. Methods of Making Mixed Metal Oxide Catalysts

It was surprisingly found that a mixed metal nano-sized catalyst of the 1) reaction product of gel oxalate co-precipitation of metals, or salts thereof, with oxalic acid, 2) reaction product of gel-precipitation of metals, or salts thereof with a precipitating agent (e.g., glycolic acid or ethylene diamine), or 3) reaction product of solid mixing of the metal precursors in the presence of an alcohol gave higher conversion to methanol in the presence of increased amounts of carbon dioxide as compared to conventional catalysts.

In the gel oxalate co-precipitation reaction, as described in the Examples and herein, metal precursors described in the Materials section can be mixed in a desired ratio with oxalic acid. In step one, two separate solutions can be prepared. The first solution can be a Cu(II) metal precursor, aluminum precursor, zinc precursor. In other instances, the first solution can include a copper(II) metal precursor, aluminum precursor, zinc precursor and M¹ (e.g., Y, Ce, Sn, Na, Mg, Bi, Gd) precursor in a solvent (e.g., alcohol, water, or a mixture thereof). Non-limiting examples of alcohol include methanol, ethanol, propanol, isopropanol, butanol, 1-butanol, or any mixture thereof. The weight ratio alcohol to the total amount metal precursor can range from 10:1 to 1.5:1, or 10;1, 9:1, 8:1, 7:1, 6:1, 5:1, 4:1, 3:1, 2:1, 1.5:1, or any weight ratio there between. In some instances, ethanol is preferred. The metal precursors can be partially or fully dissolved in the solvent. In a preferred instance, the metal precursors are fully dissolved in the solvent. The second solution can include oxalic acid dissolved in alcohol. The alcohol used to dissolve the oxalic acid can be the same or different than the alcohol used to dissolve the metal precursor. In a particular instance the same alcohol is used for both solutions. The wt. % of oxalic acid can be 10 to 120 wt. %, or 10 wt. %, 20 wt. %, 30 wt. %, 40 wt. %, 50 wt. %, 60 wt. %, 70 wt. %, 80 wt. % 90 wt. %, 100 wt. %, 110 wt. %, 120 wt. % or any value or range there between. In step two, the two solutions can be mixed slowly at room temperature (e.g., 20 to 30° C.) under vigorous agitation. By way of example, solution one can be added to solution two, or vise a versa, over a period of 1 to 10 minutes under agitation at 100 to 5000 rpms. During mixing a precipitate can form. Without wishing to be bound by theory, it is believed that the oxalic acid acts as a structure directing agent and inhibits agglomeration of particles, thereby allowing nano-sized particles of metal/oxalate precursor material to be formed. Due to their nano-size, the resulting catalyst particles can have higher electron density at the active (metal) sites. It is also believed that the use of oxalic acid reduces the number of low coordination sites (edges and corners) on the surface of the catalyst. In step 3, the metal/oxalate precipitate can be collected by standard techniques, such as decanting, filtration, or centrifuging. In a one aspect, the precipitate is centrifuged at a range from about 3000 rpm to about 7000 rpm, from about 4000 rpm to about 6000 rpm, and preferably about 5000 rpm for anywhere between 10 minutes and 30 minutes, preferably 15 minutes. The collected metal/oxalate material can be dried at temperature from about 100° C. to about 120° C., preferably 110° C. for a desired amount of time (e.g., 12 hours, 15 hours, or overnight) to obtain a dried metal/oxalate material (catalyst precursor). In step 4, the catalyst precursor can be calcined for 2 to 6 hours at a temperature of 250 to 450° C., preferably for 4 hours at a temperature of 350° C. in the presence of an oxygen source (e.g., air) to obtain a nano-sized mixed metal oxide catalyst of the general formula [CuZnAlM¹]Oₙ where n is determined by the oxidation states of the other elements and M¹ is as defined above.

The nano-sized mixed metal oxide catalysts can be obtained by a solid mixing method. In the solid mixing metal, as described in the Examples and herein, metal precursors described in the Materials section can be mixed in a desired ratio with oxalic acid and an alcohol under vigorous agitation at room temperature (e.g., 20° C. to 35° C.). Sufficient alcohol can be added to dissolve the metals and oxalic acid. Upon mixing, a precipitate forms. The precipitate can be dried at the catalyst precursor can be calcined for 2 to 6 hours at a temperature of 100 to 120° C., or 110° C. for 4 to 10 hours and then calcined at a temperature of 350° C. in the presence of oxygen (e.g., air) to obtain a nano-sized mixed metal oxide catalyst of the general formula [CuZnAlM¹]Oₙ where n is determined by the oxidation states of the other elements and M¹ is as defined above.

In some embodiments, the mixed metal oxide catalysts of the present invention can be made by a precipitating/gel method using a precipitating agent other than oxalic acid. Non-limiting examples of precipitating agents include glycolic acid, amines, diamines (e.g., ethylene diamine), aromatic amines (pyridine), and the like. In some instances, ethylene diamine or glycolic acid is used as the precipitating agent.

In a glycolic acid co-precipitation method, as described in the Examples and herein, metal precursors described in the Materials section can be mixed in a desired ratio with glycolic acid. In step one, an aqueous solution of the metal precursors can be prepared. The aqueous solution can be a copper(II) metal precursor, aluminum precursor, zinc precursor dissolved in a solvent (e.g., water, alcohol, or a mixture thereof). In other instances, the first solution can include a copper(II) metal precursor, aluminum precursor, zinc precursor and M¹ (e.g., Y, Ce, Sn, Na, Mg, Bi, Gd) precursor dissolved in water. The metal precursors can be partially or fully dissolved in the solvent. In step two (2), glycolic acid solution (e.g., 40 to 60 wt. %, 45 to 55 wt. %, or 50 wt. % glycolic acid) can be added to the metal precursor solution. The pH of the solution can be then adjusted to 7.0 to 8.0, preferably 7.2 to 7.5, or 7.0, 7.1, 7.2, 7.3, 7.4, 7.5, 7.6, 7.7, 7.8, 7.9, 8.0, or any range or value there. Compounds that can be used to adjust the pH include any base capable of deprotonating the carboxylic acid functionality of the glycolic acid (e.g., pKa of 3.83). A non-limiting example of such a base can include ammonium hydroxide. The pH of the solution is maintained below a pH that promotes formation of metal hydroxides. Adjustment of the pH promotes formation of the glycolate anion, which can complex with the metal of the metal precursor. For example, ammonium hydroxide can be added to the solution to adjust the pH to about 5. Ammonium carbonate can then be added to the solution in a controlled fashion to exchange one glycolate with one carbonate. After the addition of the ammonium carbonate, the pH can then be adjusted to the desired pH (e.g., pH of 7.0 to 8.0). The formation of the glycolate/metal complex can be observed by a color change in the solution. In step three (3), after the addition of the precipitating solution, the solution can be heated at a first temperature (e.g., 50° C. to 70° C., or 55° C. to 65° C., or 60° C.) for a desired amount of time (e.g., 20 to 40 minutes, or 30 minutes) and then heated to a second temperature (e.g., 70° C. to 100° C., or 10° C. above the first temperature) to remove the water from the solution and form a gel. The collected metal/glycolate material can be dried at temperature from about 100° C. to about 170° C. in a two-stage heating process. The metal/glycolate material can be heated to about 100° C. to 120° C., preferably 120° C. for a desired amount of time (e.g., 2 hours, 5 hours, or 3 hours) and then the temperature can be raised to 150° C. to 170° C., or 160° C. to obtain a dried mixed metal/glycolate powder. The dried powder can be reduced in size (e.g., ground to fine powder) and then calcined for 2 to 6 hours at a temperature of 250° C. to 450° C., preferably for 4 hours at a temperature of 400° C. in the presence of oxygen (e.g., air) to obtain a nano-sized mixed metal oxide catalyst of the general formula [CuZnAlM¹]Oₙ where n is determined by the oxidation states of the other elements and M¹ is as defined above.

In instances where an amine or diamine is used as the precipitating agent (e.g., ethylene diamine), the precipitating agent can added to the metal precursor solution described above (step (a)) as a neat solution or fully dissolved in water or a polar solvent (e.g., alcohol). The pH of the solution can be measured and adjusted with more base, if necessary, to bring the pH to 7.0 to 8.0. In step 3, after the addition of the precipitating solution, the solution can be heated at a first temperature (e.g., 50° C. to 70° C., or 55° C. to 65° C., or 60° C.) for a desired amount of time (e.g., 20 to 40 minutes, or 30 minutes) and then heated to a second temperature (e.g., 70° C. to 100° C., or 10° C. above the first temperature) to remove the water from the solution and form a gel. The collected metal/ethylene diamine material can be dried at temperature from about 100° C. to about 170° C. in a two-stage heating process. The metal/ethylene diamine material can be heated to about 100° C. to 120° C., preferably 120° C. for a desired amount of time (e.g., 2 hours, 5 hours, or 3 hours) and then the temperature can be raised to 150° C. to 170° C., or 160° C. to obtain a dried mixed metal/ethylene diamine powder. The dried powder can be reduced in size (e.g., ground to fine powder) and then calcined for 2 to 6 hours at a temperature of 250° C. to 450° C., preferably for 4 hours at a temperature of 400° C. in the presence of oxygen source (e.g., air) to obtain a nano-sized mixed metal oxide catalyst of the general formula [CuZnAlM¹]Oₙ where n is determined by the oxidation states of the other elements and M¹ is as defined above.

As described, the nano-sized mixed metal catalysts of the present invention are prepared under oxidative conditions (i.e. calcination) and the metals included in the heterogeneous catalyst are present in higher oxidation states, for example as oxides. Prior to being used as hydrogenation catalysts for the direct conversion of CO₂ to methanol, the catalyst can be treated under reducing conditions to convert some or all of the metals to a lower, more active, oxidation state (e.g., a zero valence). By way of example, the prepared mixed metal oxide catalysts of the current invention can be subjected to reducing conditions (e.g., a gaseous hydrogen stream) within the reactor or separately at a temperature ranging from about 220° C. to about 300° C., from about 250° C. to about 290° C. and preferably around 270° C. under 10% to 50% H₂ in Ar, 20% to 40% H₂ in Ar, and preferably 25% H₂ in Ar for 1 h to 3 h, and preferably 2 h.

The catalysts of the present invention can be pressed into pellets (e.g., combined with a binder or other materials and extruded to form pellets) sized from 2 to mm n height and 2-10 mm in diameter. The pellets can be reduced in size (e.g., crushed and sieved) to a desired particle size appropriate for loading into a reactor. For example, particle sizes of the pellets can be 100 μm to about 600 μm, from about 200 μm to about 500 μm, and preferably between 250 μm and 425 μm. These pellets contain nano-sized particles of the mixed metal catalysts.

### C. Hydrogen/Carbon Dioxide or Hydrogen/Carbon Monoxide/Carbon Dioxide Stream

Carbon dioxide, hydrogen, carbon monoxide or mixtures thereof can be obtained from various sources. In one non-limiting instance, the carbon dioxide can be obtained from a waste or recycle gas stream (e.g., from a plant on the same site, like for example from ammonia synthesis) or after recovering the carbon dioxide from a gas stream. A benefit of recycling such carbon dioxide as a starting material in the process of the invention is that it can reduce the amount of carbon dioxide emitted to the atmosphere (e.g., from a chemical production site). The hydrogen may be from various sources, including streams coming from other chemical processes, like water splitting (e.g., photocatalysis, electrolysis, or the like), syngas production, ethane cracking, methanol synthesis, or conversion of methane to aromatics. In a particular aspect, the reactant gases used in the current embodiments can be derived from syngas that includes CO₂ or from addition of CO₂ to the syngas. The H₂/CO₂ or H₂/(CO+CO₂) reactant gas streams ratio for the hydrogenation reaction can range from 1 to 5, from 1.5 to 3.5, and preferably from 1.9 to 2.9. In one instance the reactant gas stream includes 30 to 80% H₂, 1 to 30% CO₂, and 0 to 60% CO, or 40 to 70% H₂, 5 to 25% CO₂, and 0 to 20% CO, preferably about 55.5% H₂, 8% CO₂, and 11.1% CO, about 55.5% H₂, 9% CO₂, and 11.1% CO, about 55.5% H₂, 10% CO₂, and 11.1% CO, about 55.5% H₂, 11% CO₂, and 11.1% CO, about 55.5% H₂, 12% CO₂, and 11.1% CO, about 55.5% H₂, 13% CO₂, and 11.1% CO, about 55.5% H₂, 14% CO₂, and 11.1% CO, about 55.5% H₂, 15% CO₂, and 11.1% CO, about 55.5% H₂, 16% CO₂, and 11.1% CO, about 55.5% H₂, 17% CO₂, and 11.1% CO, about 55.5% H₂, 18% CO₂, and 11.1% CO. In another instance the reactant gas stream includes 1% to 20% CO₂, preferably 5% to 15% CO₂, and more preferably 8% to 12% CO₂. In some examples, the remainder of the reactant gas stream can include another gas or gases provided the gas or gases are inert, such as argon (Ar) or nitrogen (N₂), and do not negatively affect the reaction. All possible percentages of CO₂+H₂+inert gas or CO₂+CO+H₂+ inert gas in the current embodiments can have the described H₂/CO₂ or H₂/(CO+CO₂) ratios herein. Preferably the reactant mixture is highly pure and substantially devoid of water or steam. In some embodiments, the carbon dioxide can be dried prior to use (e.g., pass through a drying media) or contains a minimal amount of or no water.

### D. Methanol Production System

Conditions sufficient for the hydrogenation of CO₂ or mixtures of CO and CO₂ to methanol include temperature, time, space velocity, and pressure. The temperature range for the hydrogenation reaction can range from about 200° C. to 300° C., from about 210° C. to 280° C., preferably from about 220° C. to about 260° C. and all ranges there between including 220° C., 225° C., 230° C., 235° C., 240° C., 245° C., 250° C., 255° C., and 260° C. The gas hourly space velocity (GHSV) for the hydrogenation reaction can range from about 2,500 h⁻¹ to about 20,000 h⁻¹, from about 3,000 h⁻¹ to about 15,000 h⁻¹, and preferably from about 4,000 h⁻¹ to about 10,000 h⁻¹. The average pressure for the hydrogenation reaction can range from about 0.1 MPa to about 10 MPa, preferably about 5 MPa to about 9 MPa or 0.1, 1, 1.5, 2, 2.5, 3, 3.5, 4, 4.5, 5, 5.5, 6, 6.5, 7, 7.5, 8, 8.5, 9 MPa and all ranges or values there between. The upper limit on pressure can be determined by the reactor used. The conditions for the hydrogenation of CO₂ or mixtures of CO and CO₂ to methanol can be varied based on the type of the reactor.

In another aspect, the reaction can be carried out over the nano-sized mixed metal heterogeneous catalyst of the current invention having the particular methanol selectivity and conversion for prolonged periods of time without changing or re-supplying new catalyst or preforming catalyst regeneration. This is due to the stability or slower deactivation of the catalysts of the present invention. Therefore, in one aspect, the reaction can be performed where a single pass methanol selectivity is 40 to 100%, preferably 50 to 90%, or more preferably from 60 to 80%, or 40%, 45%, 50%, 55%, 60%, 65%, 70%, 75%, 80%, 85%, 90%, 95%, 100% after 300 hours or more time on stream (TOS). In another aspect the single pass CO₂ conversion is about 10% to 45%, about 15% to 40%, and preferably 20% to 35% at 200° C. to 260° C. and 40 to 100 bar and the methanol single space-time yield (STY) is 600 g/L.cat.h to 900 g/L.cat.h at 200 to 260° C. and 40 to 100 bar. The catalysts of the present invention can remain 90 to 99% active, preferably 94 to 98% active, after 800 hours of TOS or longer. The method can further include collecting or storing the produced methanol along with using the produced methanol as a feed source, solvent or a commercial product. Prior to use, the catalyst can be subjected to reducing conditions to convert the copper oxide and the other metals in the catalyst to a lower valance state (e.g., Cu⁺² to Cu⁺¹ and Cu⁰ species). A non-limiting example of reducing conditions includes flowing a gaseous stream that includes hydrogen gas (e.g., a H₂ and Argon gas stream) at a temperature of 250 to 280° C. for a period of time (e.g., 1, 2, or 3 hours).

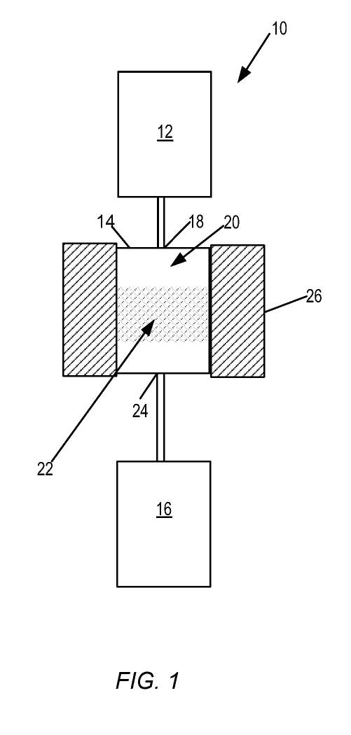

Referring to FIG. 1, a system 10 is illustrated, which can be used to convert a reactant gas stream of carbon dioxide (CO₂) and hydrogen (H₂) or carbon dioxide (CO₂) and hydrogen (H₂) and carbon monoxide (CO) into methanol using the mixed metal oxide catalysts of the present invention. The system 10 can include a reactant gas source 12, a reactor 14, and a collection device 16. The reactant gas source 12 can be configured to be in fluid communication with the reactor 14 via an inlet 18 on the reactor. As explained above, the reactant gas source 12 can be configured such that it regulates the amount of reactant feed entering the reactor 14. As shown, the reactant gas source 12 is one unit feeding into one inlet 18, however, it should be understood that the number of inlets and/or separate feed sources can be adjusted to reactor sizes and/or configurations. The reactor 14 can include a reaction zone 20 having the mixed metal oxide catalyst 22 of the present invention. The reactor can include various automated and/or manual controllers, valves, heat exchangers, gauges, etc. necessary for the operation of the reactor. The reactor can have the necessary insulation and/or heat exchangers to heat or cool the reactor as necessary. The amounts of the reactant feed and the mixed metal oxide catalyst 22 used can be modified as desired to achieve a given amount of product produced by the system 10. Non-limiting examples of continuous flow reactors that can be used include fixed-bed reactors, fluidized reactors, bubbling bed reactors, slurry reactors, rotating kiln reactors, moving bed reactors or any combinations thereof when two or more reactors are used. The reactor 14 can include an outlet 24 configured to be in fluid communication with the reaction zone and configured to remove a first product stream comprising methanol from the reaction zone 20. Reaction zone 20 can further include the reactant feed and the first product stream. The products produced can include methanol, carbon monoxide, and water. In some aspects, the catalyst can be included in the product stream. The collection device 16 can be in fluid communication with the reactor 14 via the outlet 24. Both the inlet 18 and the outlet 24 can be opened and closed as desired. The collection device 16 can be configured to store, further process, or transfer desired reaction products (e.g., methanol) for other uses. In a non-limiting example, collection device can be a separation unit or a series of separation units that are capable of separating the liquid components from the gaseous components from the product stream. By way of example, the methanol and water can be condensed from the gas stream. Any unreacted reactant gas can be recycled and included in the reactant feed to further maximize the overall conversion of CO₂ to methanol, increases the efficiency and commercial value of the CO₂ to methanol conversion process of the present invention. The water can be removed from the methanol using known drying/separation methods for the removal of water from methanol. The resulting methanol can be sold, stored or used in other processing units as a feed source. Still further, the system 10 can also include a heating/cooling source 26. The heating/cooling source 26 can be configured to heat or cool the reaction zone 20 to a temperature sufficient (e.g., 220 to 260° C.) to convert CO₂ in the reactant feed to methanol. Non-limiting examples of a heating/cooling source 20 can be a temperature controlled furnace or an external, electrical heating block, heating coils, or a heat exchanger.

### EXAMPLES

The present invention will be described in greater detail by way of specific examples. The following examples are offered for illustrative purposes only, and are not intended to limit the invention in any manner. Those of skill in the art will readily recognize a variety of noncritical parameters which can be changed or modified to yield essentially the same results.

### Catalyst Preparation

### Example 1A through Example 1J

### Synthesis of Cu/Zn/Al Gel Oxalate Co-Precipitation

The catalyst with an atomic ratio of copper, zinc and aluminum of 60:30:10 were prepared by gel oxalate co-precipitation using oxalic acid. Two separate solutions were prepared (i) a mixture of Copper (II) nitrate trihydrate, zinc nitrate hexahydrate, and aluminum nitrate nonahydrate were dissolved in ethanol. Table 1 lists the amounts of metal precursor, type and amount of alcohol, and amount of oxalic acid. In all cases the metal precursor was dissolved in the alcohol and the oxalic acid was dissolved in alcohol. The two solutions were mixed slowly at room temperature under vigorous stirring, except for Example 1F, which was heated at 45° C. during the mixing and for Example 1H in which the oxalic acid was added dropwise. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

TABLE 1

| Exam- | Cu | Zn | Al | | |
|---|---|---|---|---|---|
| ple | species | species | species | Alcohol | Oxalic acid |
| No. | (g) | (g) | (g) | (mL) | (g) (alcohol) |
| 1A | 6.84 | 4.09 | 4.17 | Ethanol | 6.37 |
| | | | | (200 ml) | (100 ethanol) |
| 1B | 11.40 | 6.82 | 6.95 | Ethanol | 10.61 |
| | | | | (200 mL) | (100 ml ethanol) |
| 1C | 11.40 | 6.82 | 6.95 | Methanol | 10.61 |
| | | | | (200 mL) | (100 ml methanol) |
| 1D | 11.40 | 6.82 | 6.95 | 2-Propanol | 10.61 |
| | | | | (200 mL) | (100 ml 2-propanol) |
| 1E | 11.40 | 6.82 | 6.95 | 1-Butanol | 10.61 |
| | | | | (200 mL) | (100 ml 1-butanol) |
| 1F | 11.40 | 6.82 | 6.95 | Ethanol | 10.61 |
| | | | | (200 mL) | (100 ml ethanol)¹ |
| 1G | 11.40 | 6.82 | 6.95 | Ethanol | 10.61 |
| | | | | (50 mL) | (50 ml ethanol) |
| 1H | 11.40 | 6.82 | 6.95 | Ethanol | 10.61 |
| | | | | (50 mL) | (50 ml ethanol)² |
| 1I | 11.40 | 6.82 | 6.95 | Ethanol | 10.61 |
| | | | | (100 mL) | (50 ml ethanol) |
| 1J | 11.40 | 6.82 | 6.95 | Ethanol | 10.61 |
| | | | | (50 mL) | (50 ml ethanol) |

¹heated to 45° C. during mixing.
²oxalic acid was added dropwise.

### Example 2

### Synthesis of Cu/Zn/Al Solid Mixing

A Cu/Zn/Al nano-sized catalyst of the present invention having an atomic ratio of copper, zinc and aluminum of 60:30:10 was prepared by a solid mix method. Cu, Zn and Al nitrates (11.40 g of copper (II) nitrate trihydrate, 6.82 g of zinc nitrate hexahydrate and 6.95 g of aluminum nitrate nonahydrate) and oxalic acid (10.61 g) were mixed in a mixer with ethanol (20 mL) for 20 min and 2000 rpm at room temperature. The formed precipitate was dried at 110° C. overnight to form the catalyst precursor. The catalyst precursor was calcined at 350° C. for 4 h to obtain the mixed metal oxide catalyst.

### Example 3

### Synthesis of Cu/Zn/Al Glycolic Acid Co-Precipitation

A Cu/Zn/Al nano-sized catalyst of the present invention having an atomic ratio of copper, zinc and aluminum of 60:30:10 was prepared by glycolate co-precipitation using an aqueous glycolic acid solution (50 wt. %). The mixture of Cu, Zn and Al nitrates (11.40 g of copper (II) nitrate trihydrate, 6.82 g of zinc nitrate hexahydrate and 6.95 g of aluminum nitrate nonahydrate) were dissolved in 100 ml water. After complete dissolution of the metal precursor, glycolic acid was added (17 mL that corresponded to a molar ratio 2.5). Ammonium hydroxide solution (20 mL) was added to adjust the pH value to about 5. Then ammonium carbonate (10 grams) was slowly added to the solution. Again ammonium hydroxide was added to reach a final pH around 7.2 to 7.5. Then heating was started with a first set point of 60° C. for 30 min then the temperature increased by 10° C. to start evaporating the water until a gel formed. The gel is dried at 120° C. (2 h) and 160° C. (4 h) respectively. The dried powder was ground and then calcined at 400° C.

### Example 4

### Synthesis of Cu/Zn/Al/Y

The catalyst with an atomic ratio of copper, zinc, aluminum and yttrium of 60:30:5:5 were prepared by gel oxalate co-precipitation using oxalic acid. Two separate solutions were prepared (i) a mixture of Cu, Zn, Al, and Y nitrates were dissolved in ethanol (11.40 g of copper (II) nitrate trihydrate, 6.82 g of zinc nitrate hexahydrate, 3.47 g of aluminum nitrate nonahydrate and 1.08 g of yttrium (III) nitrate hexahydrate), and (ii) oxalic acid (11.04 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 5

### Synthesis of Cu/Zn/Zr/Al/Ce

The catalyst with an atomic ratio of copper, zinc, aluminum and cerium of 60:30:5:5 were prepared by gel oxalate co-precipitation using oxalic acid. Two separate solutions were prepared (i) a mixture of Cu, Zn, Zr, Al, and Ce, nitrates were dissolved in ethanol (11.40 g of copper (II) nitrate trihydrate, 6.82 g of zinc nitrate hexahydrate, 3.47 g of aluminum nitrate nonahydrate and 0.77 g of cerium (III) nitrate hexahydrate), and (ii) oxalic acid (10.88 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 6

### Synthesis of Cu/Zn/Al/Sn

The catalyst with an atomic ratio of copper, zinc, aluminum and tin of 60:30:9:1 were prepared by gel oxalate co-precipitation using oxalic acid. Two separate solutions were prepared (i) a mixture of Cu, Zn, Al, and Sn nitrates were dissolved in ethanol (11.40 g of copper (II) nitrate trihydrate, 6.82 g of zinc nitrate hexahydrate, 6.25 g of aluminum nitrate nonahydrate and 0.1 g of tin(II) acetate), and (ii) oxalic acid (13.20 g) was dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 7

### Synthesis of Cu/Zn/Al/Sn

The catalyst with an atomic ratio of copper, zinc, aluminum and tin of 60:30:5:3 were prepared by gel oxalate co-precipitation using oxalic acid. Two separate solutions were prepared (i) a mixture of Cu, Zn, Al, and Sn nitrates dissolved in ethanol (11.40 g of copper (II) nitrate trihydrate, 6.82 g of zinc nitrate hexahydrate, 4.86 g of aluminum nitrate nonahydrate and 0.3 g of tin(II) acetate) and (ii) oxalic acid (12.77 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 8

### Synthesis of Cu/Zn/Al/Sn

The catalyst with an atomic ratio of copper, zinc, aluminum and tin of 60:30:7:3 were prepared by gel oxalate co-precipitation using oxalic acid. Two separate solutions were prepared (i) a mixture of Cu, Zn, Al, and Sn, nitrates dissolved in ethanol (11.40 g of copper (II) nitrate trihydrate, 6.82 g of zinc nitrate hexahydrate, 3.47 g of aluminum nitrate nonahydrate and 0.5 g of tin(II) acetate), and (ii) oxalic acid (12.33 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 9

### Synthesis of Cu/Zn/Al/Mg

The catalyst with an atomic ratio of copper, zinc, aluminum and magnesium of 60:30:9:1 were prepared by gel oxalate co-precipitation using oxalic acid. Two separate solutions were prepared (i) a mixture of Cu, Zn, Al, and Mg nitrates dissolved in ethanol (11.40 g of copper (II) nitrate trihydrate, 6.82 g of zinc nitrate hexahydrate, 6.25 g of aluminum nitrate nonahydrate and 0.53 g of magnesium nitrate hexahydrate) and (ii) oxalic acid (13.45 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 10

### Synthesis of Cu/Zn/Al/Mg

The catalyst with an atomic ratio of copper, zinc, aluminum and magnesium of 60:30:7:3 were prepared by gel oxalate co-precipitation using oxalic acid. Two separate solutions were prepared (i) a mixture of Cu, Zn, Al and Mg nitrates dissolved in ethanol (11.40 g of copper (II) nitrate trihydrate, 6.82 g of zinc nitrate hexahydrate, 4.86 g of aluminum nitrate nonahydrate and 1.58 g of magnesium nitrate hexahydrate) and (ii) oxalic acid (13.51 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 11

### Synthesis of Cu/Zn/Al/Mg

The catalyst with an atomic ratio of copper, zinc, aluminum and magnesium of 60:30:5:5 were prepared by gel oxalate co-precipitation using oxalic acid. Two separate solutions were prepared (i) a mixture of Cu, Zn, Al, and Mg nitrates dissolved in ethanol (11.40 g of copper (II) nitrate trihydrate, 6.82 g of zinc nitrate hexahydrate, 3.47 g of aluminum nitrate nonahydrate and 2.64 g of magnesium nitrate hexahydrate), and (ii) oxalic acid (13.57 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 12

### Synthesis of Cu/Zn/Al/Na

The catalyst with an atomic ratio of copper, zinc, aluminum and sodium of 60:30:9:1 were prepared by gel oxalate co-precipitation using oxalic acid. Two separate solutions were prepared (i) a mixture of Cu, Zn, Al, Na nitrates dissolved in ethanol (11.40 g of copper (II) nitrate trihydrate, 6.82 g of zinc nitrate hexahydrate, 6.25 g of aluminum nitrate nonahydrate and 0.18 g of sodium nitrate), and (ii) oxalic acid (13.46 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 13

### Synthesis of Cu/Zn/Al/Na

The catalyst with an atomic ratio of copper, zinc, aluminum and sodium of 60:30:7:3 were prepared by gel oxalate co-precipitation using oxalic acid. Two separate solutions were prepared (i) a mixture of Cu, Zn, Al, and Na nitrates dissolved in ethanol (11.40 g of copper (II) nitrate trihydrate, 6.82 g of zinc nitrate hexahydrate, 4.86 g of aluminum nitrate nonahydrate and 0.55 g of sodium nitrate), and (ii) oxalic acid (13.56 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 14

### Synthesis of Cu/Zn/Al/Na

The catalyst with an atomic ratio of copper, zinc, aluminum and sodium of 60:30:5:5 were prepared by gel oxalate co-precipitation using oxalic acid. Two separate solutions were prepared (i) a mixture of Cu, Zn, Al, and Na nitrates dissolved in ethanol (11.40 g of copper (II) nitrate trihydrate, 6.82 g of zinc nitrate hexahydrate, 3.47 g of aluminum nitrate nonahydrate and 0.92 g of sodium nitrate) and (ii) oxalic acid (13.66 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 15

### Synthesis of Cu/Zn/Al/Bi

The catalyst with an atomic ratio of copper, zinc, aluminum and bismuth of 60:30:9:1 were prepared by gel oxalate co-precipitation using oxalic acid. Two separate solutions were prepared (i) a mixture of Cu, Zn, Al, Bi nitrates dissolved in ethanol (11.40 g of copper (II) nitrate trihydrate, 6.82 g of zinc nitrate hexahydrate, 6.25 g of aluminum nitrate nonahydrate and 0.12 g of bismuth(III) nitrate pentahydrate), and (ii) oxalic acid (13.17 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 16

### Synthesis of Cu/Zn/Al/Bi

The catalyst with an atomic ratio of copper, zinc, aluminum and bismuth of 60:30:5:3 were prepared by gel oxalate co-precipitation using 20% excess of oxalic acid. Two separate solutions were prepared (i) a mixture of Cu, Zn, Al, and Bi nitrates dissolved in ethanol (11.40 g of copper (II) nitrate trihydrate, 6.82 g of zinc nitrate hexahydrate, 4.86 g of aluminum nitrate nonahydrate and 0.35 g of bismuth(III) nitrate pentahydrate), and (ii) oxalic acid (12.68 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 17

### Synthesis of Cu/Zn/Al/Bi

The catalyst with an atomic ratio of copper, zinc, aluminum and bismuth of 60:30:5:5 were prepared by gel oxalate co-precipitation using oxalic acid. Two separate solutions were prepared (i) a mixture of Cu, Zn, Al, and Bi nitrates dissolved in ethanol (11.40 g of copper (II) nitrate trihydrate, 6.82 g of zinc nitrate hexahydrate, 3.47 g of aluminum nitrate nonahydrate and 0.58 g of bismuth(III) nitrate pentahydrate), and (ii) oxalic acid (12.20 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 18

### Synthesis of Cu/Zn/Al/Gd

The catalyst with an atomic ratio of copper, zinc, aluminum and gadolinium of 60:30:9:1 were prepared by gel oxalate co-precipitation using oxalic acid. Two separate solutions were prepared (i) a mixture of Cu, Zn, Al, and Gd nitrates dissolved in ethanol (11.40 g of copper (II) nitrate trihydrate, 6.82 g of zinc nitrate hexahydrate, 6.25 g of aluminum nitrate nonahydrate and 0.14 g of gadolinium(III) nitrate hexahydrate) and (ii) oxalic acid (13.18 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 19

### Synthesis of Cu/Zn/Al/Gd

The catalyst with an atomic ratio of copper, zinc, aluminum and gadolinium of 60:30:7:3 were prepared by gel oxalate co-precipitation using oxalic acid. Two separate solutions were prepared (i) a mixture of Cu, Zn, Al, and Gd nitrates dissolved in ethanol (11.40 g of copper (II) nitrate trihydrate, 6.82 g of zinc nitrate hexahydrate, 4.86 g of aluminum nitrate nonahydrate and 0.43 g of gadolinium(III) nitrate hexahydrate), and (ii) oxalic acid (12.72 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 20

### Synthesis of Cu/Zn/Al/Gd

The catalyst with an atomic ratio of copper, zinc, aluminum and gadolinium of 60:30:5:5 were prepared by gel oxalate co-precipitation using oxalic acid. Two separate solutions were prepared (i) a mixture of Cu, Zn, Al, and Gd nitrates dissolved in ethanol (11.40 g of copper (II) nitrate trihydrate, 6.82 g of zinc nitrate hexahydrate, 3.47 g of aluminum nitrate nonahydrate and 0.72 g of gadolinium(III) nitrate hexahydrate) and (ii) oxalic acid (12.25 g) dissolved in ethanol. The two solutions were mixed slowly at room temperature under vigorous stirring. The formed precipitate was separated by centrifuge with 5000 rpm for 15 minutes, and then dried at 110° C. overnight to form the catalyst precursor. The catalyst precursor was calcined at 350° C. for 4 h to obtain the mixed metal catalyst of the present invention.

### Example 21

### Catalyst Testing

General Procedure. Catalyst testing was performed in a high throughput reactor system provided by HTE Company, Germany. The reactors are fixed bed type reactor with 0.5 cm inner diameter and 60 cm in length. Gas flow rates were regulated using Brooks SLA5800 mass flow controllers. Reactor pressure was maintained by restricted capillary before and after the reactor. The reactor temperature was maintained by an external, electrical heating block. The effluent of the reactors is connected to Agilent gas chromatography (GC) 7890 A for online gas analysis. The GC consists of two TCDs to analyze the permanent gases and one FID to analyze hydrocarbons. The column which were used to separate the chemical components are Restek Alumina Bond and Molecular Sieve. Catalysts of the present invention were pressed into pellets then crushed and sieved between 250-425 μm. A 0.54 ml of catalyst sieved fraction was placed on top of inert material inside the reactor. The commercial catalyst was used as received. Prior to the reaction test, the catalyst was reduced at 270° C. under 25 vol. % H₂ in Ar for 2 h. A mixture of 30 to 80 vol. % H₂, 0 vol. % to 60 vol. % CO and 1 vol. % to 60 vol. % CO₂, with gas hourly space velocity (GHSV)=2500, 5000, or 10000 h⁻¹ was introduced into the reactor at 30 and 40 bar and different reaction temperature (e.g., 200, 220, 230, and 240° C.). Argon was used as an internal standard for GC analysis. Methanol space time yield was calculated as follows:

STY (gₘₑₜₕₐₙₒₗ/L.hr)=X_{CO2}×S_{CH3OH}×(Mole Fed per Gram of Catalyst mol_{CO2}/ml_{cat}.hr)×32(gₘₑₜₕₐₙₒₗ/mol)×1000(ml/L)

STY=Space time yield; X_{CO2}=CO₂ Conversion; S_{CH3OH}=Methanol selectivity

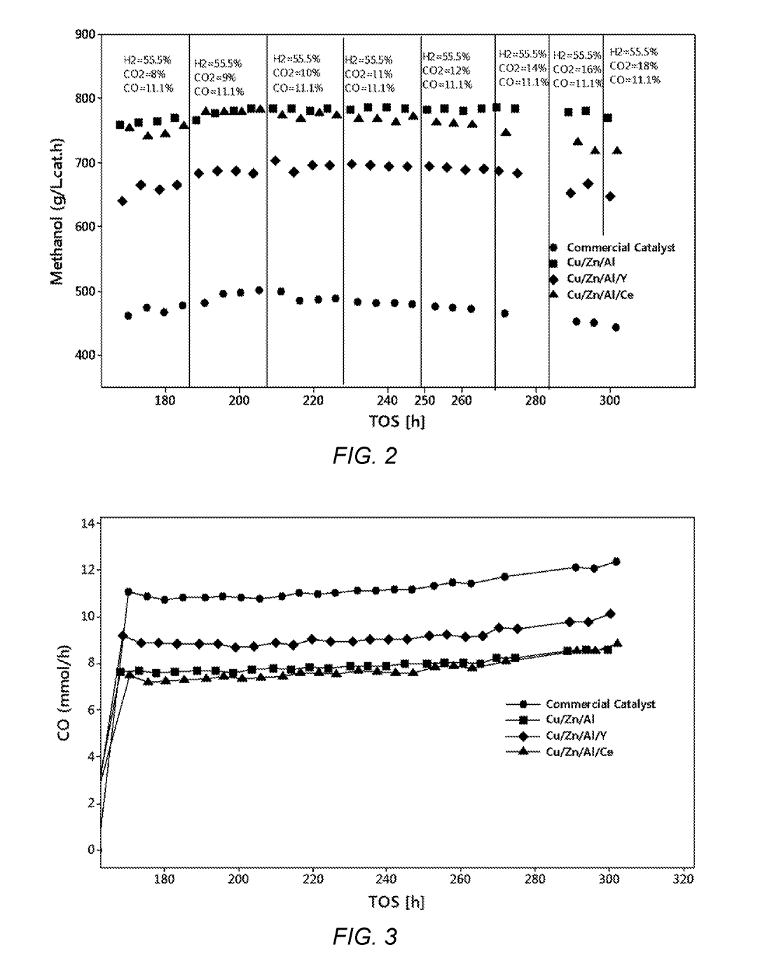

Results. FIG. 2 shows the methanol space-time yield (STY) as a function of time on stream (TOS) during the CO₂ addition into a H₂/CO mixture at 240° C., 40 bar and 5000 h⁻¹ over various nano-sized mixed heterogeneous metal catalysts prepared for Examples 1A, 4, and 5, and the commercial catalyst. The methanol yield over the commercial catalyst decreased when the CO₂ addition was more than 10 vol. % CO₂, while the catalysts of the present invention did not show a decrease in methanol conversion until the CO₂ addition was greater than 15 vol. %. The Cu/Zn/Al/Y started declining at 16 vol. % CO₂ while the Cu/Zn/Al started declining at 18 vol. % CO₂.

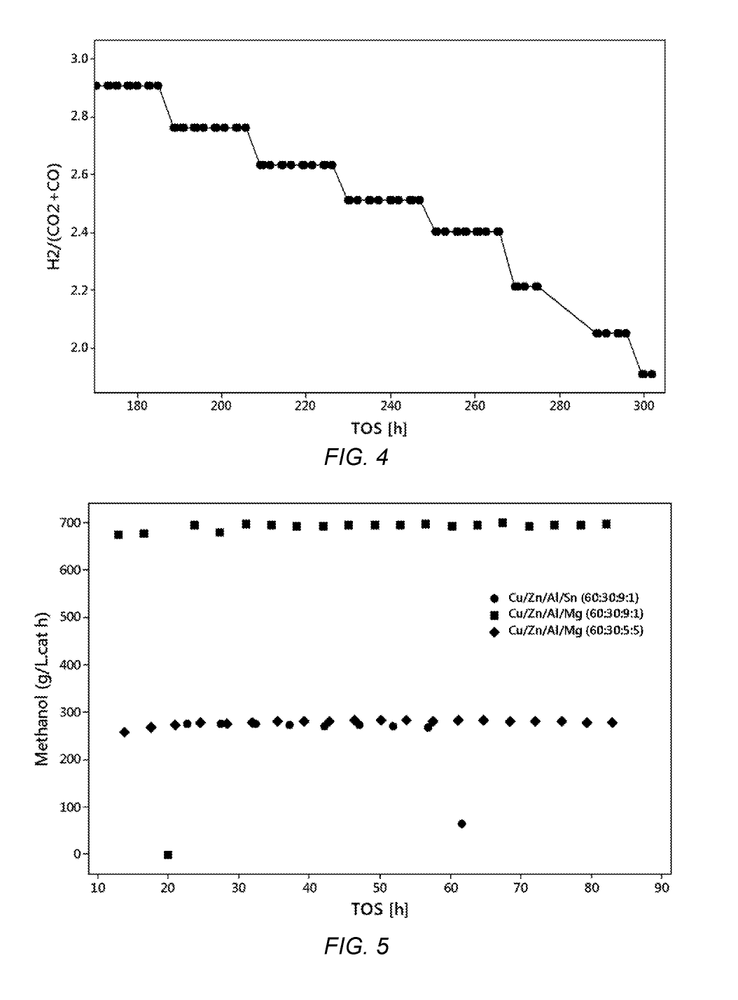

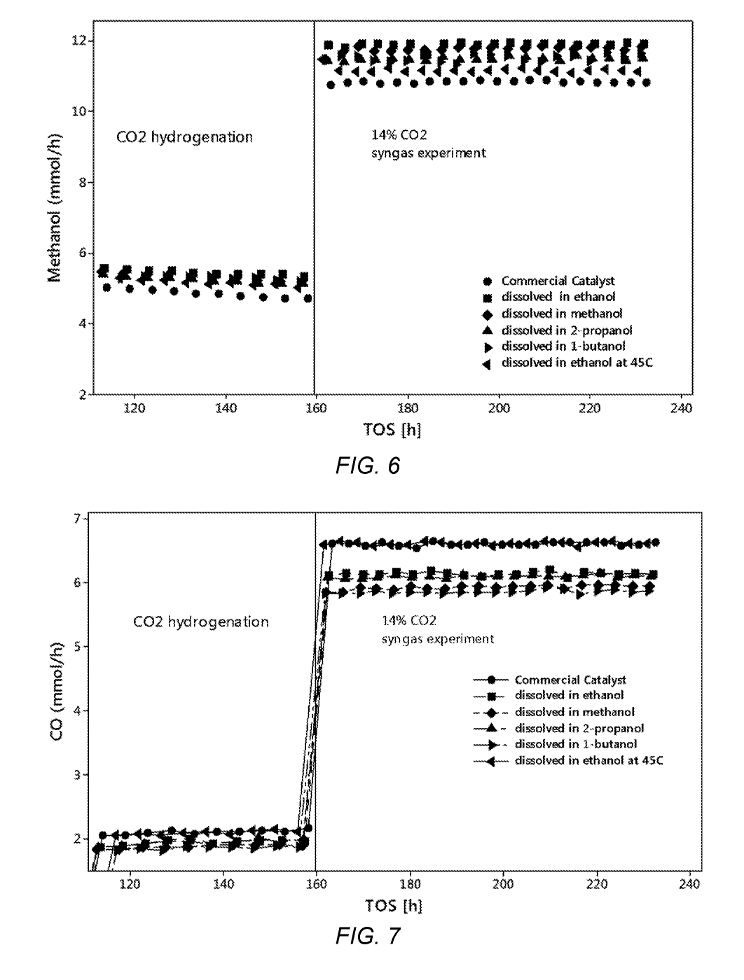

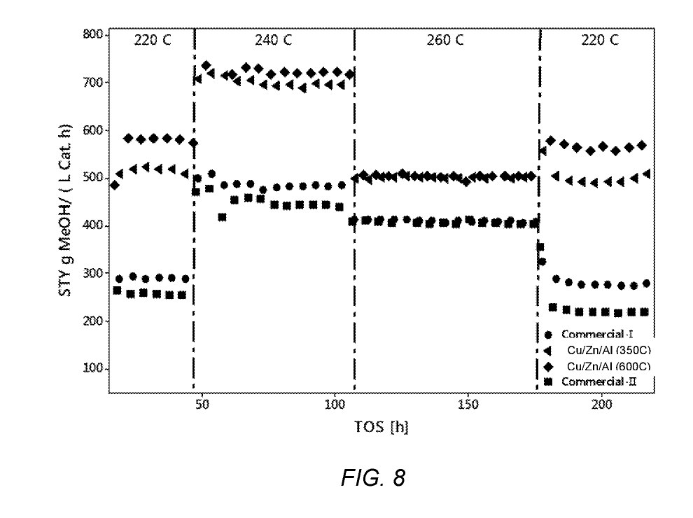

FIG. 3 shows the molar flow rate of CO during the CO₂ addition into a H₂/CO mixture at 240° C., 40 bar and 5000 h⁻¹ over various catalysts prepared for Examples 1A, 4, and 5, and the commercial catalyst. From the data, it was determined that the commercial catalyst produced more CO (about 11 vol. %) than the catalysts of Examples 1-3 (about 7 to 9 vol. %). Without wishing to be bound by theory, it is believed that the CO was produced by reverse water gas shift reaction (See, reaction scheme 2 above). FIG. 4 shows a graph of the H₂/(CO₂+CO) versus time on stream for Examples 1A, 4, and 5. As shown in FIG. 4, the highest ratio of H₂/(CO₂+CO) used was 2.9 and the lowest was 1.9. FIG. 5 shows the methanol space-time yield (STY) as a function of time on stream (TOS) during the CO₂ addition into a H₂/CO mixture at 240° C., 40 bar and 5000 h⁻¹ over various nano-sized mixed heterogeneous metal catalysts prepared for Examples 6, 9 and 11. From the data in FIGS. 2 and 5, the nano-sized heterogeneous mixed metal catalysts from Examples 1A, 4, 5, and 9 had a higher methanol productivity as compared to commercial catalyst. FIG. 6 shows the molar flow rate of methanol from hydrogenation of CO₂ and after the addition of 14 vol. % CO₂ into a H₂/CO mixture at 240° C., 40 bar and 5000 h⁻¹ over various catalysts prepared for Examples 1B-1F, and the commercial catalyst. FIG. 7 shows the molar flow rate of carbon monoxide from hydrogenation of CO₂ and after the addition of 14 vol. % CO₂ into a H₂/CO mixture at 240° C., 40 bar and 5000 h⁻¹ over various catalysts prepared for Examples 1B-1F, and the commercial catalyst. The rate of methanol production was greater than 10 mmol/h, when 14 vol. % of CO₂ was present in the reaction mixture. From the data, all of the catalysts of the present invention showed greater CO₂ hydrogenation and produced more methanol from the H₂/CO and CO₂ mixtures with increased amounts of CO₂ than the commercial catalyst.

FIG. 8 shows the methanol space-time yield (STY) as a function of time on stream (TOS) for addition of a feed stream having 14 vol % CO₂ /balance Ar to a mixture of gas having 55 vol. % H₂/11 vol. % CO at 220° C. and 240° C., 40 bar (4.0 MPa) and 5000 h⁻¹ over various nano-sized mixed heterogeneous metal catalysts prepared for an Cu/Zn/Al oxide catalysts having a Cu/Zn/Al atomic ratio of 67:29:4 (made using the gel-oxalate co-precipitation above and calcined at 350° C. and 600° C.), and two commercial catalysts (commercial catalyst I and commercial catalyst II). From the data, the nano-sized heterogeneous mixed metal catalysts had a higher methanol productivity as compared to commercial catalyst, with the highest yield being produced at a temperature of at 240° C.

### Example 22

### Catalyst Characterization

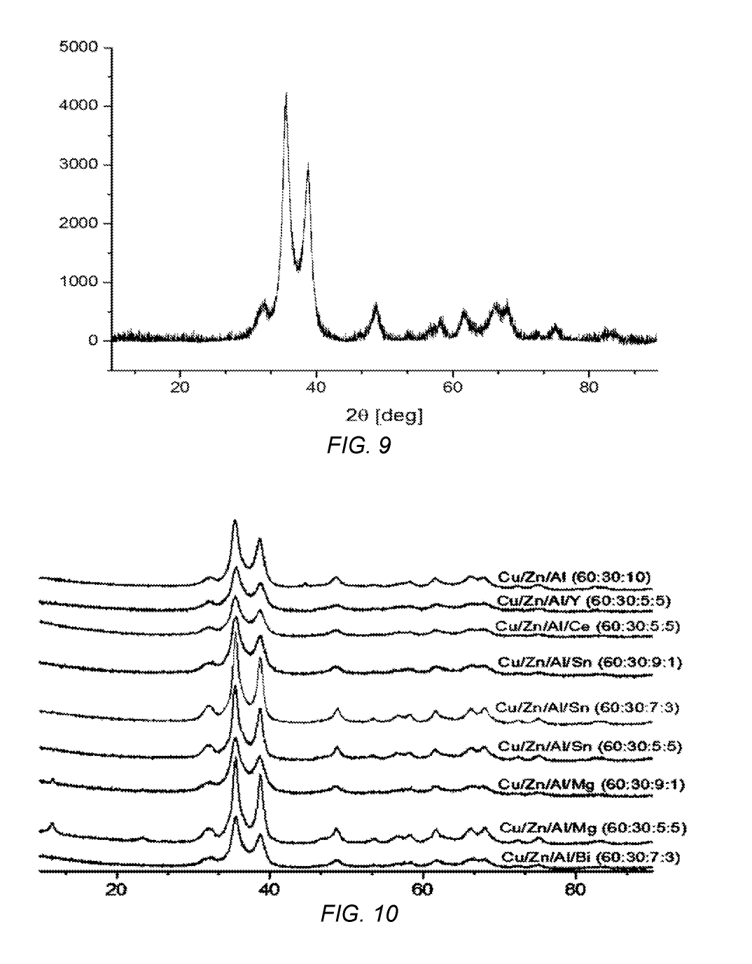

X-Ray Diffraction (XRD) Analysis. XRD pattern of the catalysts were obtained using Empyrean from PANalytical using a nickel-filtered CuKα X-ray source, a convergence mirror and a PIXcelld detector. The scanning rate was 0.01° over the range between 5° and 80° 2θ. The XRD pattern for Example 1A is depicted in FIG. 9. FIG. 10 shows the XRD patterns for the catalysts of Examples 1A, 4-9, 11 and 15. The physical characteristics of the catalyst of Example 1A and the commercial catalyst are listed in Table 2. Physical characteristics of the catalyst of Examples 1A, 4-9, 11, 15, 16, 18, and 20 are listed in Table 3.

Surface Area Analysis. Catalyst BET surface area was determined using a Micromeritics ASAP 2020 instrument. Copper surface area was determined by N₂O pulse titration technique.

TABLE 2

| | | Comparative Catalyst |
|---|---|---|
| Property | Example 1A | (Commercial) |
| Particle Size (nm) | 8 | 10 |
| BET Surface Area (m²/g) | 70 | 92 |
| Cu Surface area (m²/g_{Cat.}) | 19.9 | 13.8 |
| Pore Volume (cm³/g) | 0.26 | 0.3 |
| Pore Diameter (nm) | 14.9 | 12.8 |
| Initial crystalline phases* | CuO and | CuO, ZnO, and |
| | ZnO | (CuZn)₆Al₂(OH)₁₆CO₃•4H₂O |

*The initial crystalline phase was determined by XRD after the catalyst has been calcined and before the catalyst is exposed to the operating conditions within a reactor.

TABLE 3

| | | Crystallite | Surface | Pore | Pore |
|---|---|---|---|---|---|
| | Exam- | size | Area | Volume | Diameter |
| Composition | ple | (nm) | (m²/g) | (cm³/g) | (nm) |
| Cu/Zn/Al | 1A | 10.1 | 70.56 | 0.259 | 14.71 |
| (60:30:10) | | | | | |
| Cu/Zn/Al/Y | 4 | 9.3 | NA | NA | NA |
| (60:30:5:5) | | | | | |
| Cu/Zn/Al/Ce | 5 | 8.9 | 78.75 | 0.221 | 11.23 |
| (60:30:5:5) | | | | | |
| Cu/Zn/Al/Sn | 6 | 8.6 | NA | NA | NA |
| (60:30:9:1) | | | | | |
| Cu/Zn/Al/Sn | 7 | 15.9 | 51.56 | 0.2 | 15.55 |
| (60:30:7:3) | | | | | |
| Cu/Zn/Al/Sn | 8 | 15.6 | NA | NA | NA |
| (60:30:5:5) | | | | | |
| Cu/Zn/Al/Mg | 9 | 8.8 | NA | NA | NA |
| (60:30:9:1) | | | | | |
| Cu/Zn/Al/Mg | 11 | 15.8 | 62.16 | 0.21 | 13.55 |
| (60:30:5:5) | | | | | |
| Cu/Zn/Al/Bi | 15 | NA | 59.34 | 0.204 | 13.81 |
| (60:30:9:1) | | | | | |
| Cu/Zn/Al/Bi | 16 | 9 | 67.2 | 0.233 | 13.9 |
| (60:30:7:3) | | | | | |
| Cu/Zn/Al/Gd | 17 | NA | 77.43 | 0.23 | 12.24 |
| (60:30:9:1) | | | | | |
| Cu/Zn/Al/Gd | 20 | NA | 73.29 | 0.189 | 10.32 |
| (60:30:5:5) | | | | | |

1. A mixed metal catalyst capable of producing methanol (CH₃OH) from hydrogen (H₂) and carbon dioxide (CO₂) or from hydrogen (H₂), carbon dioxide (CO₂) and carbon monoxide (CO), the catalyst having a general formula of:
[CuₐZn_{b}Al_{c}Md¹]Oₙ
where a is 20 to 80, b is 15 to 60, c is 1 to 25, d is 0 to 15 and n is determined by the oxidation states of the other elements; and
where M¹ is yttrium (Y), cerium (Ce), tin (Sn), sodium (Na), magnesium (Mg), bismuth (Bi), or gadolinium (Gd).

2. The mixed metal catalyst of claim 1, wherein M¹ is Y.

3. The mixed metal catalyst of claim 1, wherein M¹ is Ce.

4. The mixed metal catalyst of claim 1, wherein M¹ is Sn.

5. The mixed metal catalyst of claim 1, wherein M¹ is Na.

6. The mixed metal catalyst of claim 1, wherein M¹ is Bi.

7. The mixed metal catalyst of claim 1, wherein M¹ is Gd.

8. The mixed metal catalyst of claim 1, wherein M¹ is Mg.

9. The mixed metal catalyst of claim 1, where in d is 0 and the catalyst has the formula of: [CuₐZn_{b}Al_{c}]Oₙ

10. The mixed metal oxide catalyst of claim 1, wherein the catalyst has a particle size of 2 to 12 nm.

11. A method of producing methanol (CH₃OH) from hydrogen (H₂) and carbon dioxide (CO₂) or from hydrogen (H₂), carbon dioxide (CO₂) and carbon monoxide (CO), the method comprising contacting a reactant gas stream that includes H₂ and CO₂ or H₂, CO₂, and CO with a nano-sized heterogeneous mixed metal catalyst of claim 1 under conditions sufficient to produce a product gas stream comprising CH₃OH.

12. The method of claim 11, wherein the ratio of H₂/(CO₂+CO) is 1.5 to 3.5, preferably 1.9 to 2.9.

13. The method of claim 11, wherein the reactant gas stream includes 30 to 80% H₂, 1 to 30% CO₂, and 0 to 60% CO, or the reactant gas stream includes 1% to 20% CO₂, preferably 5% to 15% CO₂, and more preferably 8% to 12% CO₂.

14. A method of making a mixed metal oxide catalyst of claim 1, the method comprising:
(a) obtaining a first solution comprising metal precursor materials that includes copper (Cu), zinc (Zn), aluminum (Al) and, optionally, M¹, where M¹ is yttrium (Y), cerium (Ce), tin (Sn), sodium (Na), bismuth (Bi), magnesium (Mg), gadolinium (Gd), or any combination thereof;
(b) obtaining a second solution comprising oxalic acid dissolved in an alcohol;
(c) mixing the first and second solution together to form a precipitate from the metal precursor materials; and
(d) drying and calcining the precipitate to obtain the mixed metal oxide catalyst.

15. The method of claim 14, wherein metal precursor materials include copper (Cu), zinc (Zn), aluminum (Al) and M¹

16. A method of making a mixed metal oxide catalyst of claim 1, the method comprising:
(a) obtaining an aqueous solution comprising metal precursor materials that includes copper (Cu), zinc (Zn), aluminum (Al) and, optionally, M¹, where M¹ is yttrium (Y), cerium (Ce), tin (Sn), sodium (Na), bismuth (Bi), magnesium (Mg), gadolinium (Gd), or any combination thereof, wherein the metal precursors are dissolved in the aqueous solution;
(b) adding a precipitating agent to the aqueous solution; and
(c) heating the aqueous solution to form a gel; and
(d) drying and calcining the gel to obtain the mixed metal oxide catalyst.

17. The method of claim 16, wherein the precipitating agent in step (b) is glycolic acid and the method further comprises adjusting the pH of the aqueous solution to 7.0 to 8.0, preferably 7.2 to 7.5, during or after the addition of the glycolic acid.

18. The method of claim 16, wherein the precipitating agent in step (b) is diethylene amine.

19. A method of making a mixed metal oxide catalyst of claim 1 to 10, the method comprising:
(a) mixing oxalic acid, an alcohol, and metal precursor materials to form a precipitate, wherein the metal precursor materials include copper (Cu), zinc (Zn), aluminum (Al) and, optionally, M¹, where M¹ is yttrium (Y), cerium (Ce), tin (Sn), sodium (Na), bismuth (Bi), magnesium (Mg), gadolinium (Gd), or any combination thereof; and
(b) drying and calcining the precipitate to obtain the mixed metal oxide catalyst.

20. The method of claim 19, wherein metal precursor materials include copper (Cu), zinc (Zn), aluminum (Al) and M¹.
